(19) United States
(12) Patent Application Publication
(10) Pub. No.: US 2013/0060017 A1
(43) Pub. Date: Mar. 7, 2013

(54) METHODS FOR SYNTHESIZING LABELED COMPOUNDS

(71) Applicants: Dennis ESHIMA, Phoenix, AZ (US); Mehmet HUSNU, Phoenix, AZ (US); Jim STONE, Alpharetta, GA (US); Brian C. KELLEY, Pataskala, OH (US)

(72) Inventors: Dennis ESHIMA, Phoenix, AZ (US); Mehmet HUSNU, Phoenix, AZ (US); Jim STONE, Alpharetta, GA (US); Brian C. KELLEY, Pataskala, OH (US)

(73) Assignee: CARDINAL HEALTH 414, LLC, Dublin, OH (US)

(21) Appl. No.: 13/550,055

(22) Filed: Jul. 16, 2012

Related U.S. Application Data
(60) Provisional application No. 61/508,408, filed on Jul. 15, 2011.
(60) Provisional application No. 61/508,294, filed on Jul. 15, 2011.

(51) Int. Cl. C07H 1/00

(52) U.S. Cl. 536/122

(57) ABSTRACT

The invention relates to methods for synthesizing compounds on solid supports. The invention also relates to synthesizing labeled compounds, such as those used in the field of radiopharmaceuticals. The invention relates to methods for synthesizing halogenated radiopharmaceuticals such as fludeoxyglucose (also known as fluorodeoxyglucose or FDG).

### RELATED APPLICATIONS

This application claims priority to U.S. Provisional Application No. 61/508,408 entitled “METHOD FOR SYNTHESIZING LABELED COMPOUNDS” filed on Jul. 15, 2011; and U.S. Provisional Application No. 61/508,294 filed on Jul. 15, 2011, titled “SYSTEMS, METHODS, AND DEVICES FOR PRODUCING, MANUFACTURING, AND CONTROL OF RADIOPHARMACEUTICALS.” Each of the above applications is incorporated by reference herein in its entirety.

### FIELD OF THE INVENTION

The invention relates to methods for synthesizing compounds on solid supports. The invention also relates to synthesizing labeled compounds, such as those used in the field of radiopharmaceuticals. The invention relates to methods for synthesizing halogenated radiopharmaceuticals such as fludeoxyglucose (also known as fluorodeoxyglucose or FDG).

### BACKGROUND OF THE INVENTION

Many compounds are synthesized by subjecting a starting compound to one or more reactions. Some of these processes involve a step of forming an intermediary compound containing one or more protecting groups. These intermediary compounds are then subjected to a deprotection reaction, wherein the protecting groups are removed and replaced with other groups to form a final compound.

In the synthesis of some compounds, solid supports, such as columns and cartridges containing, for example, grains, membranes, sheets and/or capillaries, are used. In such processes, a starting compound and/or intermediary compounds are present on an intact solid support, and typically, removal from the solid support only occurs when the final compound is formed. In some synthesis processes known in the art, every step of the synthesis process occurs on the intact solid support. Radiopharmaceuticals are often synthesized on solid supports in this manner.

Radiopharmaceuticals are used in nuclear medicine for a variety of functions, including the use as tracers for the diagnosis and treatment of many diseases. Radiopharmaceuticals are used in imaging and functional studies of the body and specifically organs and tissues such as the brain, myocardium, thyroid, gall bladder, liver, kidney, skeleton/bone, blood, and tumors. An example of a radiopharmaceutical is FDG.

FDG, which is also known as fludeoxyglucose, fluorodeoxyglucose, [¹⁸F]FDG, and 2-deoxy-2-(¹⁸F)fluoro-D-glucose, is a radiopharmaceutical which is widely used in nuclear medicine for diagnostic studies, such as those using Positron Emission Tomography (PET) body scanning. FDG contains the positron-emitting radioactive isotope of fluorine-18. After the injection of FDG in a patient, a PET scanner forms images of the FDG in the body, and these images can be used to diagnose a number of medical indications. FDG has the following chemical structure:

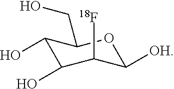 [SMILES: OCC1OC(O)C([18F])C(O)C1O]

Processes for the synthesis of FDG are described in the following references, which are each incorporated by reference in their entirety: Hamacher et al. (“Efficient Stereospecific Synthesis of No-Carrier-Added 2-[¹⁸F]-Fluoro-2-Deoxy-D-Glucose Using Aminopolyether Supported Nucleophilic Substitution,” J. Nucl. Med. 27:235-238, 1986); Mulholland (“Simple Rapid Hydrolysis of Acetyl Protecting Groups in the FDG Synthesis Using Cation Exchange Resins,” Nucl. Med. Biol. Vol. 22, No. 1, pp. 19-23, 1995); and Füchtner et al. (“Basic Hydrolysis of 2-[¹⁸F]fluoro-1,3,4,6-tetra-O-acetyl-D-glucose in the Preparation of 2-[¹⁸F]fluoro-2-deoxy-D-glucose,” Appl. Radiat. Isol. Vol. 47, No. 1, pp. 61-66, 1996).

U.S. Pat. No. 6,172,207 to Damhaut et al. discloses methods for synthesizing FDG, involving the trapping of a protected precursor compound, tetraacetylfluoroglucose (FTAG), on a solid support. Deprotection (hydrolysis) of the FTAG compound with sodium hydroxide (elimination of the protecting groups) occurs directly on the solid support comprised in a column or cartridge. The resulting deprotected compound, FDG, is removed from the solid support which is comprised in the column or cartridge.

There is a need in the art for alternative synthesis processes wherein the reaction step of forming a final compound does not occur on a solid support. There is also a need in the art for alternative synthesis methods which do not utilize traditional, intact solid supports. There is also a need in the art for an alternative method of synthesizing radiopharmaceuticals, such as FDG.

The method of the present invention may be used in with the systems, method, and devices described in Applicants' co-pending U.S. Provisional Application No. 61/508,294 entitled “SYSTEMS, METHODS, AND DEVICES FOR PRODUCING, MANUFACTURING, AND CONTROL OF RADIOPHARMACEUTICALS” filed on Jul. 15, 2011, the entirety of which is incorporated by reference herein.

Additional advantages and novel features relating to aspects of the present invention will be set forth in part in the description that follows, and in part will become more apparent to those skilled in the art upon examination of the following or upon learning by practice thereof.

All references cited herein are incorporated by reference in their entirety.

### SUMMARY OF THE INVENTION

The invention relates to methods for synthesizing labeled compounds, especially in the field of radiopharmaceuticals. One aspect of the claimed invention relates to a method comprising the steps of: labeling a protected precursor compound with a labeling agent to form a labeled protected compound; contacting the labeled protected compound with a solid support; removing the labeled protected compound from the solid support with a removing agent; and deprotecting the labeled protected compound with a deprotecting agent to form a labeled deprotected compound (final labeled compound).

Another aspect of the claimed invention relates to a method comprising the steps of: labeling a protected precursor compound with a labeling agent to form a labeled protected compound; contacting the labeled protected compound with a medium containing two or more pieces of solid support or other media; deprotecting the labeled protected compound in the medium with a deprotecting agent to form a labeled deprotected compound; and removing the labeled deprotected compound (final labeled compound) from the solid support or other media with a removing agent.

### DETAILED DESCRIPTION OF THE INVENTION

The invention relates to methods for synthesizing compounds, wherein part of the synthesis method occurs on solid supports. Examples of such compounds include but are not limited to labeled compounds, such as radiopharmaceuticals. Examples of labeled compounds include but are not limited to radiopharmaceuticals, such as FDG. In some preferred embodiments, the labeled compound is FDG. Other compounds include compounds which are formed by one or more deprotection processes.

One aspect of the claimed invention relates to a method comprising the steps of: labeling a protected precursor compound with a labeling agent to form a labeled protected compound; contacting the labeled protected compound with a solid support; removing the labeled protected compound from the solid support with a removing agent; and deprotecting the labeled protected compound with a deprotecting agent to form a labeled deprotected compound (final labeled compound).

In some embodiments, the protected precursor compound is a compound containing a protecting group. The term “protecting group” includes, but is not limited to, groups such as acetyls, ethers, esters, thioesters, thioethers, imides, amides, carbamates, N-alkyles, N-aryles, N-heteroderivatives, cetals, acetals, ketals, acylals, benzoyls, benzyls, trityls, pivaloyls, sulfonamides, dithianes, and tetrahydropyranyls. In some preferred embodiments, the protecting group is an acetal group. In some embodiments where the final labeled compound is FDG, the labeled protected compound is mannose triflate, which has the following structure, wherein Ac is CH₃CO— and Tf is CF₃SO₂—:

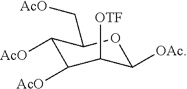

The labeling agent used in the present invention may be a radioactive group, such as a radioactive halogen. In some embodiments wherein the final labeled compound is FDG, the labeling agent is ¹⁸F. The labeling agent may be prepared by any method known in the art. In embodiments where the final labeled compound is FDG, the labeling agent may be used to form a labeled protected compound such tetraacetylfluoroglucose, or FTAG, which has the following structure:

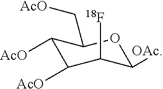 [SMILES: CC(=O)OCC1OC(OC(C)=O)C([18F])C(OC(C)=O)C1OC(C)=O]

The labeled protected compound may be formed from the protected precursor compound with a phase transfer catalyst (such as, but not limited to 4,7,13,16,21,24-Hexaoxa-1,10-diazabicyclo[8.8.8]-hexacosane, or KRYPTOFIX® 222) and a solvent (such as, but not limited to acetonitrile).

The labeled protected compound may be contacted with a solid support. A solid support is typically a porous solid which may be found in a column or cartridge. The solid support may be or normal, reverse, or intermediate polarity phase type, and it may be of an ion exchange phase type or a mixture of ion exchange phase type with normal or reverse phases. The solid support may be in the form of grains, membranes, sheets and/or capillaries. A column or cartridge may be, but is not limited to, any type of stationary phase conditioning which may be used in chromatography. The labeled protected compound (such as FTAG) may be contacted with the solid support by passing a liquid comprising the labeled protected compound through the column or cartridge. The labeled protected compound then may become trapped in the column or cartridge, which contains the solid support, and separated from other materials which may be found in the liquid.

The labeled protected compound may then be separated from the solid support in the column or cartridge by a removing agent. The term “removing agent” includes any compound or mixtures of compounds which assist in the removal of a compound from a solid support. Examples include but are not limited to solvents, such as ethanol or any other alcohol. The labeled protected compound may be separated from the solid support by immersing the solid support with a solution comprising the removing agent for a period of time. The solution comprising the removed labeled protected compound and the removing agent may then be placed in a chamber or other container. A deprotecting agent may then contact the solution comprising the removed labeled protected compound. The term “deprotecting agent” includes any compound or mixture of compounds which removes the protecting groups, for example, through hydrolysis. Examples include, but are not limited to, bases or acids, such as the following: sodium hydroxide (NaOH) and hydrogen chloride (HCl). The deprotecting agent may be provided in a solution, such as an aqueous media. The deprotecting agent may be contacted with the solution comprising the removed labeled protected compound for a period of time. The deprotection of the labeled protected compound leads to the formation of a final labeled compound. Purification methods known in the art may be used to purify and isolate the final labeled compound, which is a labeled deprotected compound. Further treatment of the final labeled compound may be conducted. In some preferred embodiments, the final labeled compound is a labeled radiopharmaceutical, such as FDG.

Another aspect of the claimed invention relates to a method comprising the steps of: labeling a protected precursor compound with a labeling agent to form a labeled protected compound; contacting the labeled protected compound with a medium containing two or more pieces of solid support or other media; deprotecting the labeled protected compound in the medium with a deprotecting agent to form a labeled deprotected compound; and removing the labeled deprotected compound from the solid support with a removing agent.

In this aspect of the claimed invention, after the formation of the labeled protected compound, the labeled protected compound may be contacted with a medium containing two or more pieces of solid support or other media. In some embodiments, the pieces of solid support are formed by cutting, crushing, or grinding a column or cartridge containing a solid support into two or more pieces. In other embodiments, the two or more pieces are of media, such as any hard solid particles such as glass beads may be used. The solid support or other media includes any materials that can hold or trap a compound, and reactions may occur on the compounds that are held or trapped therein. In some embodiments, the column or cartridge is crushed or ground into multiple small pieces. In some embodiments, the pieces of solid support or media are placed in a liquid medium and into a chamber or other vessel, and the labeled protected compound (such as FTAG), which is also in a liquid medium, the same or different, is added to the chamber for a period of time, causing the labeled protected compound to bind to or become trapped in the pieces of solid support or media. In some embodiments where media such as glass beads are used, the media can be placed in the chamber loosely. A deprotecting agent may then be added to the chamber, which comprises the pieces of solid support and the labeled protected compound, and deprotection occurs while the labeled protected compound is bound to or trapped onto the solid support. The deprotection may lead to the formation of the final labeled compound (labeled deprotected compound), which is bound to the solid support. In some embodiments, the chamber may then be drained, leaving the solid support, which is then contacted with a removing agent to remove or separate the final labeled compound from the solid support.

In some embodiments, the chamber is a closed chamber. The use of a closed chamber may provide some advantages. For example, in some embodiments, reaction speed and efficiency may be improved, and less media or pieces of solid support may be needed. In some embodiments, the chamber may contain a drain with a multiway valve, wherein the drain can be blocked to trap the pieces of solid support or media in the chamber. In some embodiments, nitrogen or another gas may be used in the process, for example, to create a fluidized bed of particles (such as pieces of solid support or media). In some embodiments, nitrogen or another gas may be introduced into the chamber, for example, through a multiway valve, and the liquid or working fluid to be processed may be introduced to the chamber while the bed of media or solid support is fluidized. There may be a mixing and contacting of the surface of the pieces of solid support or media, wherein the intended interaction (for example, deprotection) is produced. In some embodiments where nitrogen or other gas is used, the flow of gas may be decreased or discontinued, and excess liquid media may be drained, while the pieces of solid support or media remain in the chamber. In some embodiments, optionally additional liquids may be introduced to the chamber, for example, to release or elute chemicals from the surface of the media. In some embodiments, fluidization of the bed may be used in other additional steps as well.

In some preferred embodiments, the final labeled compound is a radiopharmaceutical, such as FDG.

### Example 1

The synthesis of fludeoxyglucose (fluorodeoxyglucose, or FDG).

FDG may be synthesized in the following steps:
Formation of tetraacetylfluoroglucose (FTAG) from mannose triflate, with ¹⁸F, and with phase catalyst KRYPTOFIX®-222 and acetonitrile
   Aqueous media containing FTAG and acetonitrile transferred through column containing solid support
   Mixture drains to waste, and FTAG is retained on solid support
   Solid support is rinsed with ethanol, and FTAG is washed into chamber
   Aqueous sodium hydroxide (NaOH) is added to the chamber and deprotection (hydrolysis) occurs, leading to the formation of FDG

### Example 2

The synthesis of fludeoxyglucose (fluorodeoxyglucose, or FDG).

FDG may be synthesized in the following steps:
Solid support is ground into multiple pieces and added to a chamber filled with a liquid medium
   Aqueous media containing tetraacetylfluoroglucose (FTAG) with ¹⁸F is added to the chamber
   Chamber is drained, leaving solid support pieces
   Aqueous sodium hydroxide is added to the chamber, and deprotection (hydrolysis) of FTAG occurs on the solid support, forming FDG on the solid support
   Chamber is drained
   Ethanol is added to the chamber to remove the FDG from the solid support.

What is claimed:

1. A method for synthesizing labeled compounds, comprising:
labeling a protected precursor compound with a labeling agent to formed a labeled protected compound;
contacting the labeled precursor compound with a solid support;
removing the labeled protected compound from the solid support with a removing agents; and
deprotecting the labeled protected compound with a deprotecting agent to form a labeled deprotected compound.

2. The method of claim 1, wherein the labeled deprotected compound is a radiopharmaceutical.

3. The method of claim 1, wherein the labeled deprotected compound is a halogenated compound.

4. The method of claim 1, wherein the labeled deprotected compound is fluorodeoxyglucose.

5. The method of claim 1, wherein the removing agent comprises a solvent.

6. The method of claim 5, wherein the solvent comprises ethanol.

7. The method of claim 1, wherein the deprotecting agent comprises an acid or a base.

8. The method of claim 1, wherein the deprotecting agent comprises sodium hydroxide or hydrogen chloride.

9. The method of claim 1, wherein the protected precursor compound comprises a protecting group selected from the group consisting of acetyls, ethers, esters, thioesters, thioethers, imides, amides, carbamates, N-alkyles, N-aryles, N-heteroderivatives, cetals, acetals, ketals, acylals, benzoyls, benzyls, trityls, pivaloyls, sulfonamides, dithianes, and tetrahydropyranyls.

10. The method of claim 1, wherein the labeled protected compound comprises mannose triflate.

11. A method comprising the steps of:
labeling a protected precursor compound with a labeling agent to form a labeled protected compound;
contacting the labeled protected compound with a medium containing two or more pieces of solid support or other media;
deprotecting the labeled protected compound in the medium with a deprotecting agent to form a labeled deprotected compound; and removing the labeled deprotected compound from the solid support or other media with a removing agent.

12. The method of claim 11, wherein the labeled deprotected compound is a radiopharmaceutical.

13. The method of claim 1, wherein the labeled deprotected compound is a halogenated compound.

14. The method of claim 1, wherein the labeled deprotected compound is fluorodeoxyglucose.

15. The method of claim 1, wherein the removing agent comprises a solvent.

16. The method of claim 5, wherein the solvent comprises ethanol.

17. The method of claim 1, wherein the deprotecting agent comprises an acid or a base.

18. The method of claim 1, wherein the deprotecting agent comprises sodium hydroxide or hydrogen chloride.

19. The method of claim 1, wherein the protected precursor compound comprises a protecting group selected from the group consisting of acetyls, ethers, esters, thioesters, thioethers, imides, amides, carbamates, N-alkyles, N-aryles, N-heteroderivatives, cetals, acetals, ketals, acylals, benzoyls, benzyls, trityls, pivaloyls, sulfonamides, dithianes, and tetrahydropyranyls.

20. The method of claim 1, wherein the labeled protected compound comprises mannose triflate.
